(19) United States
(12) Patent Application Publication
(10) Pub. No.: US 2025/0144083 A1
(43) Pub. Date: May 8, 2025

(54) METHODS AND COMPOSITIONS FOR TREATING CANCER IN TAXANE-RESISTANT PATIENTS

(71) Applicant: MODRA PHARMACEUTICALS B.V., Amsterdam (NL)

(72) Inventors: Eric VAN DER PUTTEN, Leiderdorp (NL); Colin FREUND, Hoboken, NJ (US)

(21) Appl. No.: 18/838,206

(22) PCT Filed: Feb. 13, 2023
(86) PCT No.: PCT/EP2023/053502

§ 371 Date: Aug. 13, 2024

(30) Foreign Application Priority Data
Feb. 14, 2022 (GB) 2201935.0

(51) Int. Cl. A61K 31/427; A61K 31/337; A61K 31/473; A61P 35/00

(52) U.S. Cl. CPC A61K 31/427; A61K 31/337; A61K 31/473; A61P 35/00

(57) ABSTRACT

Methods and compositions are provided for treating cancer in a taxane-resistant patient, comprising administering an effective dose of a taxane simultaneously or sequentially with a P-gp inhibitor, such as ritonavir or a pharmaceutically acceptable salt thereof. Also provided are methods and compositions for reversing taxane resistance in a patient, comprising administering an effective dose of a P-gp inhibitor, such as ritonavir or a pharmaceutically acceptable salt thereof.

### TECHNICAL FIELD

The present invention relates to chemotherapy of tumours using taxanes. More particularly, it relates to methods and compositions for treating cancer in taxane-resistant patients using a combination of a P-gp inhibitor, such as ritonavir, and a taxane.

### BACKGROUND OF THE INVENTION

Treatments of cancers involve a wide range of treatment. Treatments include surgery, radiation therapy, chemotherapy, immunotherapy and cell therapy. Often, cancer treatments include a combination of different modes of treatments, comprising combinations of different therapeutic agents. As part of first line chemotherapy, taxanes, such as docetaxel or cabazitaxel, are used widely in the treatment of various cancers.

Docetaxel is a cytotoxic agent. Its main mode of action is understood to involve interference with microtubule assembly and disassembly, resulting in inhibiting mitotic cell division. The recommended dosage is a three-weekly intravenous administration, with a dose in the range of 75-100 mg/m² of body surface area. Docetaxel is used in the treatment of a variety of cancers, which include breast, lung, prostate, gastric, head and neck, and ovarian cancer. Docetaxel may be administered intravenously as a chemotherapeutic agent and is approved and in use for the treatment of a variety of solid tumors. Variability has been observed in patients with regard to response to treatment. Improved means and methods for utilizing docetaxel in the treatment of cancer have been proposed. In particular, docetaxel may be administered in combination with a cytochrome P450 isoenzyme CYP3A inhibitor, having an improved safety profile of docetaxel as compared with the standard of care treatment for docetaxel.

For example, WO 2009/027644 A2 provides a pharmaceutical composition and methods for the treatment of neoplastic disease comprising the combination of a taxane and a CYP3A4 inhibitor. WO 2020/127606 A1 provides cancer treatment using docetaxel by controlling plasma levels. WO 2020/127607 A1 provides a combination treatment for solid tumors using docetaxel and a CYP3A inhibitor.

However, while having the potential to benefit patients, improving life expectancy and quality of life, the sustained use of taxanes, such as docetaxel or cabazitaxel, may lead to the formation of taxane-resistance in patients, causing the effectiveness of the taxane in treating cancer to become limited. Moreover, some patients may be refractory to a taxane ab initio for example due to having naturally higher resistance. It is therefore highly desirable to provide a new means of treating cancer in taxane-resistant patients.

### SUMMARY OF THE INVENTION

Surprisingly, the present inventors have found that resistance in a patient to treatment with a taxane can be mitigated, substantially overcome, at least partially reversed, or substantially reversed by administration of a P-gp inhibitor, namely ritonavir. Ritonavir may also be used to sensitize or re-sensitize cells to a taxane. Examples of such taxanes may include docetaxel, cabazitaxel, paclitaxel, pharmaceutically acceptable salts or esters thereof, and combinations thereof, such as for the treatment of cancer. Examples of such cancers include solid tumours, gastric cancer, breast cancer, head and neck cancer, lung cancer, and prostate cancer.

In a first embodiment, the present invention provides a method for treating cancer in a taxane-resistant patient, comprising administering an effective dose of a taxane simultaneously or sequentially with ritonavir or a pharmaceutically acceptable salt thereof.

In a second embodiment, the present invention provides a method for reversing taxane resistance in a patient, comprising administering an effective dose of ritonavir or a pharmaceutically acceptable salt thereof.

In a third embodiment, the present invention provides a taxane for use in a combination therapy with ritonavir, or a pharmaceutically acceptable salt thereof, for treating cancer in a taxane-resistant patient.

In a fourth embodiment, the present invention provides ritonavir, or a pharmaceutically acceptable salt thereof, for use in reversing taxane resistance in a patient.

In a fifth embodiment, the present invention provides a method for sensitizing or re-sensitizing cells to a taxane in the treatment of cancer, comprising administering an effective dose of ritonavir, or a pharmaceutically acceptable salt thereof.

In a sixth embodiment, the present invention provides ritonavir, or a pharmaceutically acceptable salt thereof, for use in sensitizing or re-sensitizing cells to a taxane in the treatment of cancer.

In a seventh embodiment, the present invention provides a kit comprising at least one pharmaceutical composition comprising a taxane and at least one pharmaceutical composition comprising a P-gp inhibitor, such as ritonavir, wherein said kit is for use in treating cancer in a taxane-resistant patient.

In an eighth embodiment, the present invention provides a method for the treatment of cancer, comprising a combination of a P-gp inhibitor, such as ritonavir, and a taxane, comprising the steps of:
determining the expression of ABCB1 (P-gp) in a patient;
optionally, comparing the expression of ABCB1 (P-gp) to a reference level;
optionally, determining the P-gp inhibitor dosage based on the expression level of ABCB1 (P-gp) determined in the subject;
administering a combination of the P-gp inhibitor and the taxane.

### DETAILED DESCRIPTION

Specific embodiments of the present invention will now be described, by way of example, with reference to the accompanying drawings, in which:

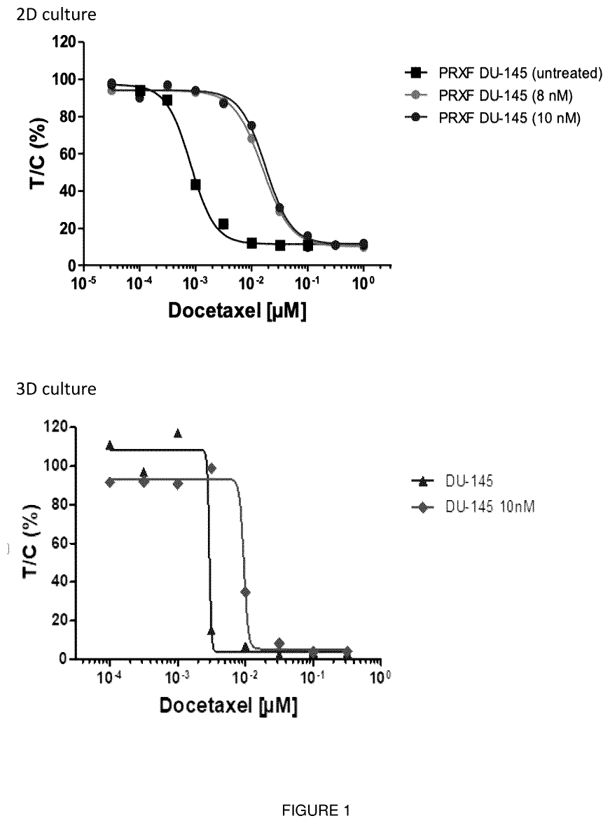

FIG. 1 shows docetaxel resistance in DU145 prostate cancer cell lines;

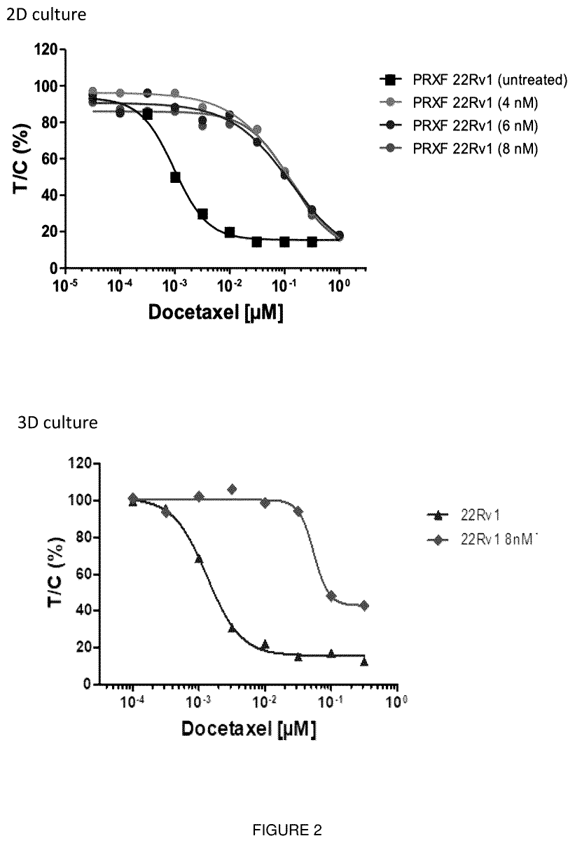

FIG. 2 shows docetaxel resistance in 22Rv1 prostate cancer cell lines;

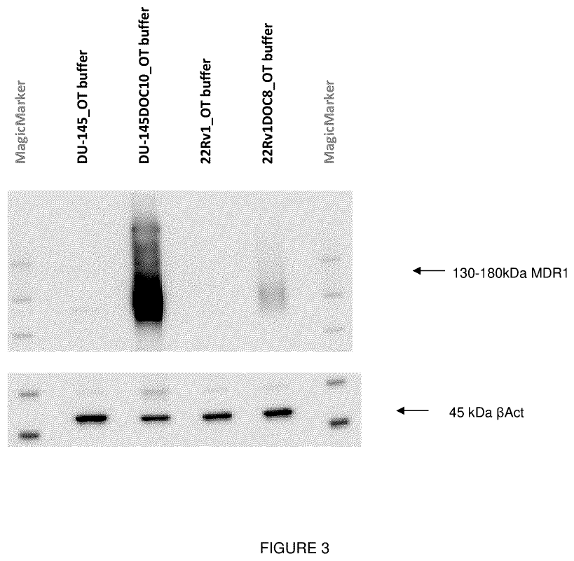

FIG. 3 shows P-gp protein expression in docetaxel resistant prostate cancer cells;

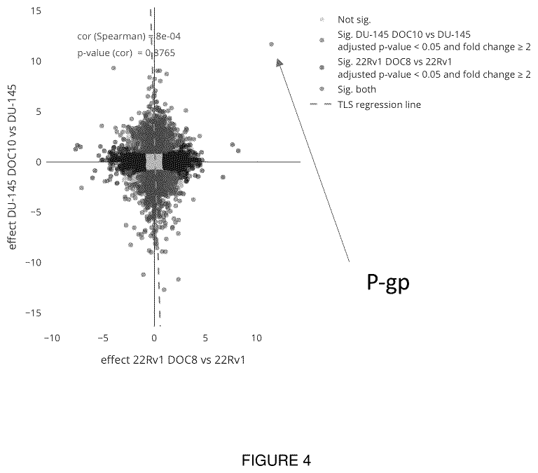

FIG. 4 shows a scatter plot comparing significant genes in DU-145 DOC10 vs DU-145 against 22Rv1 DOC8 vs 22Rv1. The upper-right panel shows genes upregulated in both pairs. The upper-left panel shows genes upregulated in DU-145 and downregulated in 22Rv1 pairs. The lower-right panel shows genes upregulated in 22Rv1 and downregulated in DU-145 pairs. The lower-left panel shows genes downregulated in both pairs;

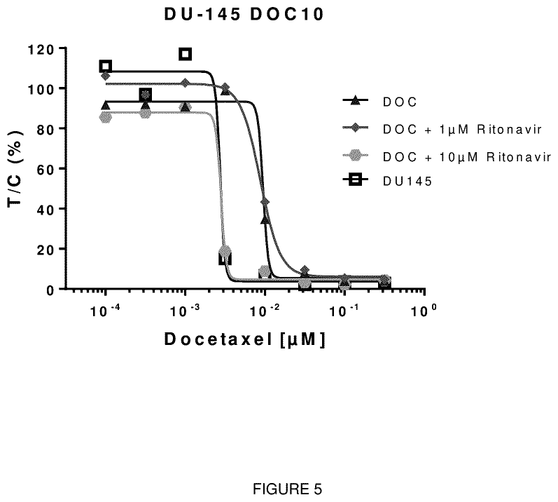

FIG. 5 shows that ritonavir reverses docetaxel resistance in castration-resistant DU-145 DOC10 prostate cancer cells (clonogenic assay);

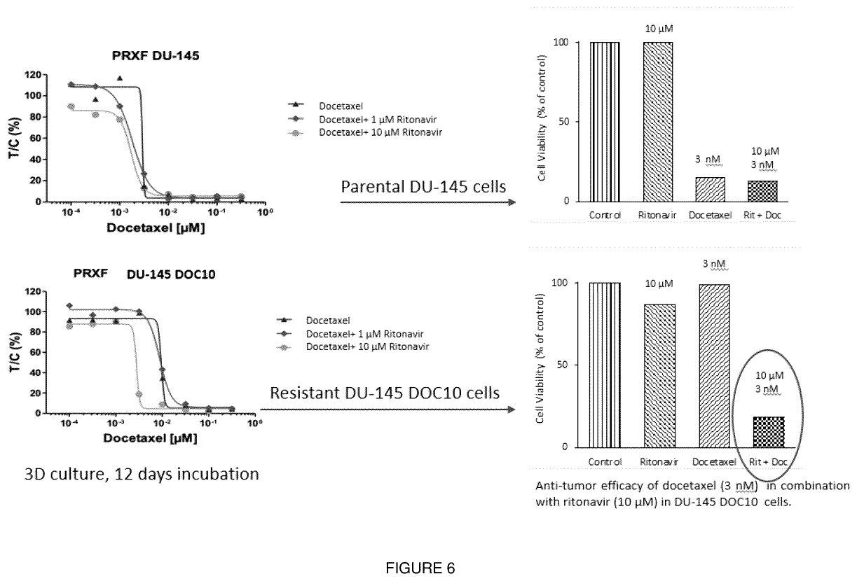

FIG. 6 shows that the combination of ritonavir and docetaxel induces synergistic effects in docetaxel resistant DU-145 DOC10 prostate cancer cells (clonogenic assay);

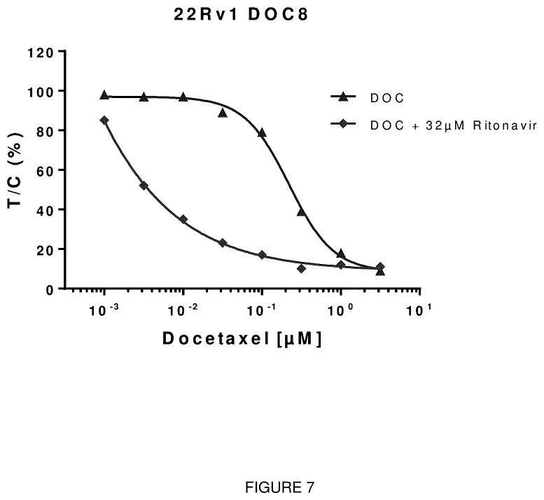

FIG. 7 shows that ritonavir also reverses docetaxel resistance in 22Rv1 DOC8 resistant prostate cancer cells;

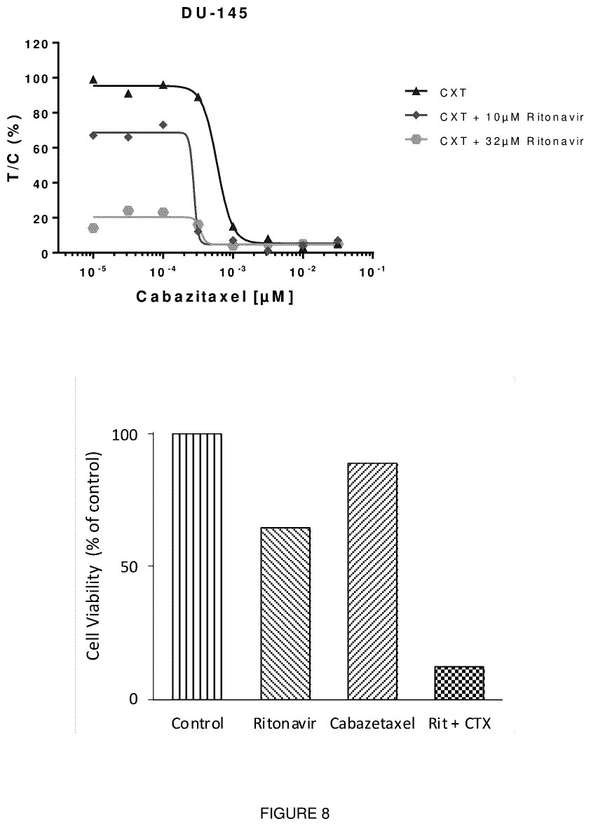

FIG. 8 shows that the combination of ritonavir and cabazitaxel induces synergistic effects in parent DU-145 prostate cancer cells;

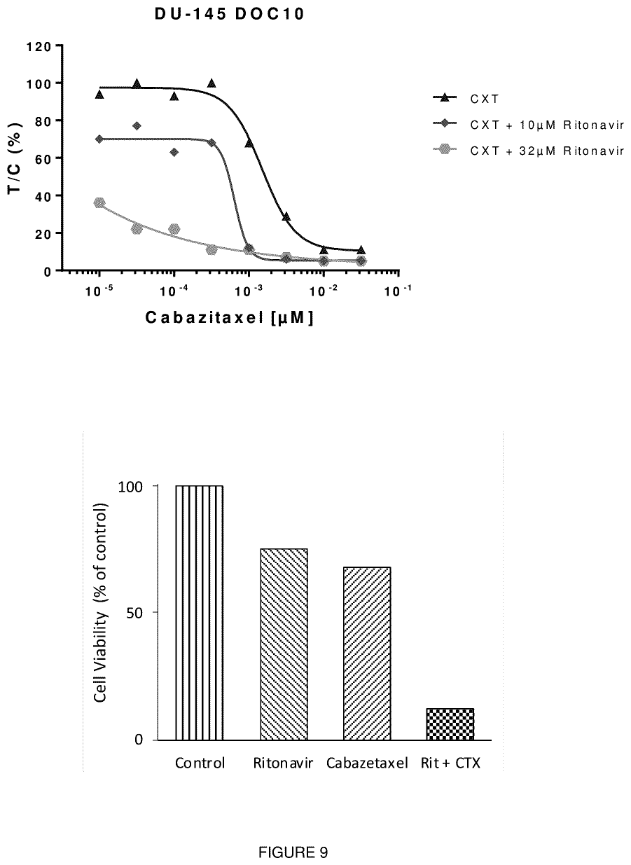

FIG. 9 shows that the combination of ritonavir and cabazitaxel induces synergistic effects in docetaxel-resistant DU-145 DOC10 prostate cancer cells;

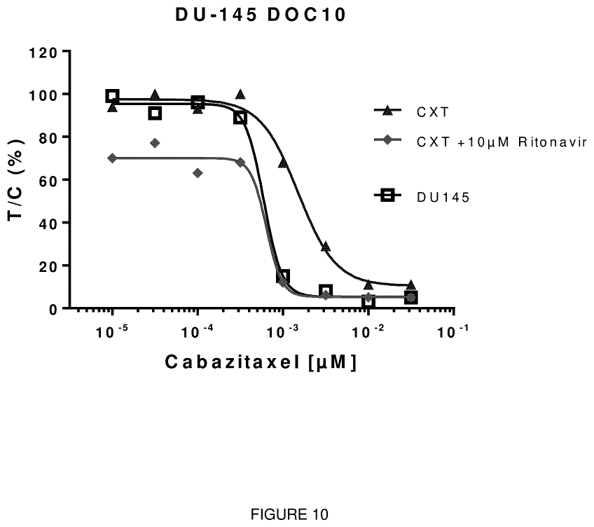

FIG. 10 shows that ritonavir reverses cabazitaxel resistance in docetaxel-resistant DU145 DOC10 prostate cancer cells;

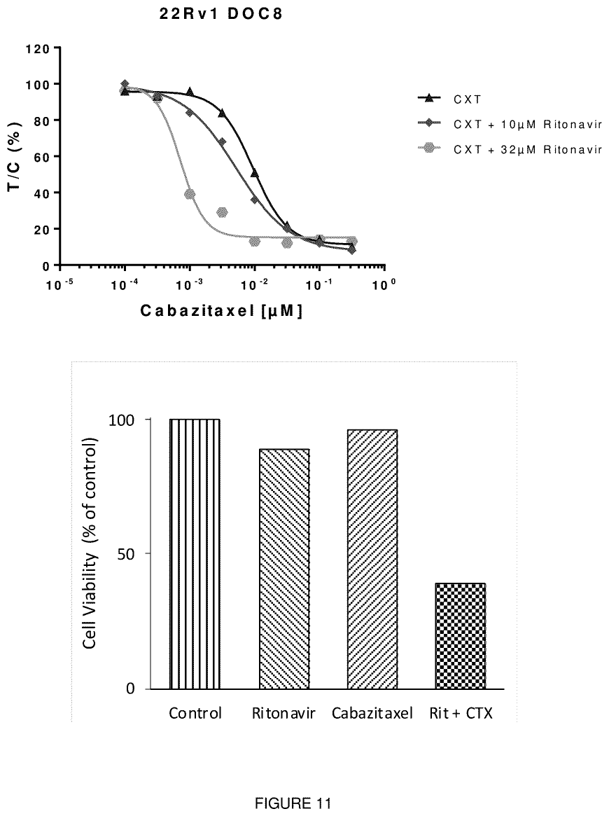

FIG. 11 shows that the combination of ritonavir and cabazitaxel induces synergistic effects in docetaxel-resistant 22Rv1 DOC8 prostate cancer cells;

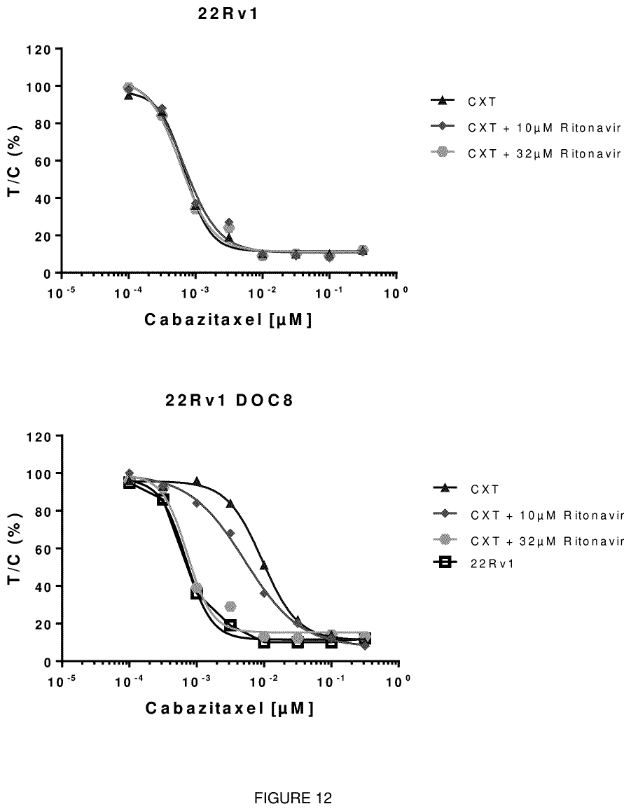

FIG. 12 shows that ritonavir reverses cabazitaxel resistance in docetaxel-resistant 22Rv1 DOC8 prostate cancer cells;

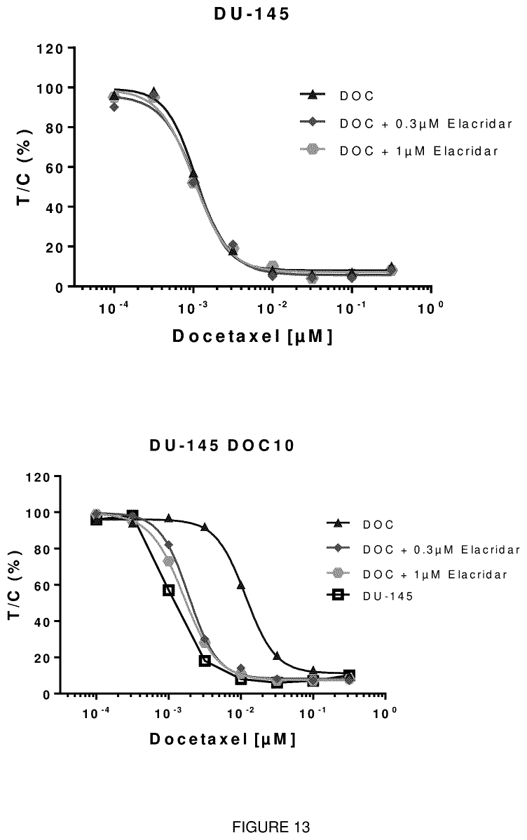

FIG. 13 shows that elacridar reverses docetaxel resistance in castration-resistant DU-145 DOC10 prostate cancer cells;

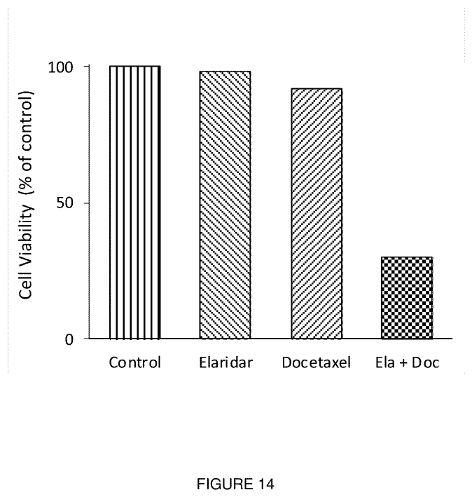

FIG. 14 shows that elacridar and docetaxel were synergistic in docetaxel resistant DU-145 DOC10 prostate cancer cells (clonogenic assay); and

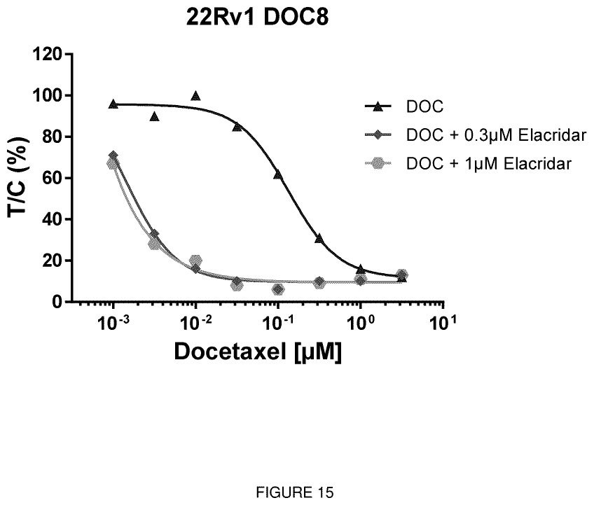

FIG. 15 shows that elacridar reverses docetaxel resistance in castration-resistant 22Rv1 DOC8 prostate cancer cells.

In a first embodiment, the present invention provides a method for treating cancer in a taxane-resistant patient, comprising administering an effective dose of a taxane simultaneously or sequentially with ritonavir or a pharmaceutically acceptable salt thereof.

In a second embodiment, the present invention provides a method for reversing taxane resistance in a patient, comprising administering an effective dose of ritonavir or a pharmaceutically acceptable salt thereof.

Herein the terms “taxane-resistant patient” and “taxane resistance” may refer to either resistance ab initio (e.g. in patients who are refractory to a taxane ab initio for example due to having naturally higher Pg-p expression) or acquired resistance (e.g. in patients where the efficacy of a taxane is reduced over time after extended use of a taxane, such as for 3 months or more, or by increasing dose of a taxane).

Acquired resistance to one taxane may also result in acquired resistance for other taxanes. For example, extended use of docetaxel (such as for 3 months or more) may result in acquired resistance to docetaxel and/or other taxanes such as cabazitaxel.

Acquired resistance may be identified in cancer cells following exposure to a taxane by observing an increase in the IC₅₀ value compared to parental untreated cells. The increase in the IC₅₀ value may for example be greater than about 10 fold increase, such as a greater than about 20 fold increase, or greater than about 100 fold increase.

Drug resistant cell lines (with ab initio or acquired resistance) may be identified by upregulated genes associated with metabolic or homeostasis pathways compared to parental untreated cells, in particular ABCB1 (P-glycoprotein). For example, ABCB1 (P-glycoprotein) may be upregulated greater than about 1000 fold, greater than about 2000 fold, or greater than about 3000 fold.

Preferably the methods are for treating cancer, wherein ritonavir, or a pharmaceutically acceptable salt thereof, is administered simultaneously or sequentially with an effective dose of a taxane.

For example, the cancer may comprise a solid tumour. For example, the cancer may be selected from the group consisting of gastric cancer, breast cancer, head and neck cancer, lung cancer, and prostate cancer. Preferably, the cancer is prostate cancer.

The methods may also comprise determining if the cancer is responsive to treatment by determining the responsiveness of a DU-145 or 22Rv1 cell line to an effective dose of a taxane simultaneously or sequentially with ritonavir or a pharmaceutically acceptable salt thereof.

Conveniently, ritonavir and/or the taxane may administered orally, which may provide improved means and methods for treating cancer with an improved safety profile as compared to a standard of care treatment for a taxane such as docetaxel, while at the same time allowing to exert control to obtain efficacious anti-tumour levels of taxane exposure. Alternatively, ritonavir and/or the taxane may be administered intravenously.

The taxane may be selected from the group consisting of docetaxel, cabazitaxel, pharmaceutically acceptable salts or esters thereof, and combinations thereof. Preferably, the taxane is docetaxel, or a pharmaceutically acceptable salt or ester thereof. Other taxanes that are envisaged include paclitaxel.

Ritonavir may be administered at a weekly dose of about 50 mg to about 1200 mg, for example about 200 mg to about 400 mg.

Alternatively, ritonavir may be administered at a daily dose of about 50 mg to about 1200 mg, for example about 100 mg to about 200 mg.

The taxane may be administered at a weekly dose of about 30 mg to about 500 mg. For example, the taxane may be docetaxel or cabazitaxel and is administered at a weekly dose of about 30 mg to about 500 mg, such as about 40 mg to about 100 mg or about 40 mg to about 50 mg.

Alternatively, the taxane may be administered at a daily dose of about 0.1 mg to about 100 mg. For example, the taxane may be docetaxel or cabazitaxel and is administered at a daily dose of about 0.1 mg to about 100 mg, such as about 40 mg to about 50 mg.

For example, ritonavir may be administered at a daily dose of about 50 mg to about 1200 mg, such as about 100 mg to about 200 mg, and the taxane may be administered at a daily dose of about 0.1 mg to about 100 mg, such as about 40 mg to about 50 mg, wherein the daily doses of ritonavir and the taxane may be taken at the same or different time of day.

In another example, ritonavir may be administered at a weekly dose of about 50 mg to about 1200 mg, such as about 200 mg to about 400 mg, and the taxane may be administered at a weekly dose of about 30 mg to about 500 mg, such as about 40 mg to about 100 mg or about 40 mg to about 50 mg, wherein the weekly doses of ritonavir and the taxane may be taken on the same or different days.

In a third embodiment, the present invention provides a taxane for use in a combination therapy with ritonavir, or a pharmaceutically acceptable salt thereof, for treating cancer in a taxane-resistant patient.

Preferably, ritonavir, or a pharmaceutically acceptable salt thereof, is administered simultaneously or sequentially with the taxane.

For example, the cancer may comprise a solid tumour. For example, the cancer may be selected from the group consisting of gastric cancer, breast cancer, head and neck cancer, lung cancer, and prostate cancer. Preferably, the cancer is prostate cancer.

The use may also comprise determining if the cancer is responsive to treatment by determining the responsiveness of a DU-145 or 22Rv1 cell line to an effective dose of the combination therapy.

Conveniently, ritonavir and/or the taxane may be administered orally, which may provide improved means and methods for treating cancer with an improved safety profile as compared to a standard of care treatment for a taxane such as docetaxel, while at the same time allowing to exert control to obtain efficacious anti-tumour levels of taxane exposure. Alternatively, ritonavir and/or the taxane may be administered intravenously.

The taxane may be selected from the group consisting of docetaxel, cabazitaxel, pharmaceutically acceptable salts or esters thereof, and combinations thereof. Preferably, the taxane is docetaxel, or a pharmaceutically acceptable salt or ester thereof. Other taxanes that are envisaged include paclitaxel.

Ritonavir may be administered at a weekly dose of about 50 mg to about 1200 mg, for example about 200 mg to about 400 mg.

Alternatively, ritonavir may be administered at a daily dose of about 50 mg to about 1200 mg, for example about 100 mg to about 200 mg.

The taxane may be administered at a weekly dose of about 30 mg to about 500 mg. For example, the taxane may be docetaxel or cabazitaxel and is administered at a weekly dose of about 30 mg to about 500 mg, such as about 40 mg to about 100 mg or about 40 mg to about 50 mg.

Alternatively, the taxane may be administered at a daily dose of about 0.1 mg to about 100 mg. For example, the taxane may be docetaxel or cabazitaxel and is administered at a daily dose of about 0.1 mg to about 100 mg, such as about 40 mg to about 50 mg.

For example, ritonavir may be administered at a daily dose of about 50 mg to about 1200 mg, such as about 100 mg to about 200 mg, and the taxane may be administered at a daily dose of about 0.1 mg to about 100 mg, such as about 40 mg to about 50 mg, wherein the daily doses of ritonavir and the taxane may be taken at the same or different time of day.

In another example, ritonavir may be administered at a weekly dose of about 50 mg to about 1200 mg, such as about 200 mg to about 400 mg, and the taxane may be administered at a weekly dose of about 30 mg to about 500 mg, such as about 40 mg to about 100 mg or about 40 mg to about 50 mg, wherein the weekly doses of ritonavir and the taxane may be taken on the same or different days.

In a fourth embodiment, the present invention provides ritonavir, or a pharmaceutically acceptable salt thereof, for use in reversing taxane resistance in a patient.

Preferably, said use is for treating cancer in a taxane-resistant patient, wherein ritonavir is administered simultaneously or sequentially with an effective dose of a taxane.

For example, the cancer may comprise a solid tumour. For example, the cancer may be selected from the group consisting of gastric cancer, breast cancer, head and neck cancer, lung cancer, and prostate cancer. Preferably, the cancer is prostate cancer.

The use may comprise determining if the cancer is responsive to treatment by determining the responsiveness of a DU-145 or 22Rv1 cell line to an effective dose of a taxane simultaneously or sequentially with ritonavir or a pharmaceutically acceptable salt thereof.

Conveniently, ritonavir and/or a taxane may be administered orally, which may provide improved means and methods for treating cancer with an improved safety profile as compared to a standard of care treatment for a taxane such as docetaxel, while at the same time allowing to exert control to obtain efficacious anti-tumour levels of taxane exposure. Alternatively, ritonavir and/or a taxane may be administered intravenously.

The taxane may be selected from the group consisting of docetaxel, cabazitaxel, pharmaceutically acceptable salts or esters thereof, and combinations thereof. Preferably, the taxane may be docetaxel, or a pharmaceutically acceptable salt or ester thereof. Other taxanes that are envisaged include paclitaxel.

Ritonavir may be administered at a weekly dose of about 50 mg to about 1200 mg, for example about 200 mg to about 400 mg.

Alternatively, ritonavir may be administered at a daily dose of about 50 mg to about 1200 mg, for example about 100 mg to about 200 mg.

The taxane may be administered at a weekly dose of about 30 mg to about 500 mg. For example, the taxane may be docetaxel or cabazitaxel and is administered at a weekly dose of about 30 mg to about 500 mg, such as about 40 mg to about 100 mg or about 40 mg to about 50 mg.

Alternatively, the taxane may be administered at a daily dose of about 0.1 mg to about 100 mg. For example, the taxane may be docetaxel or cabazitaxel and is administered at a daily dose of about 0.1 mg to about 100 mg, such as about 40 mg to about 50 mg.

For example, ritonavir may be administered at a daily dose of about 50 mg to about 1200 mg, such as about 100 mg to about 200 mg, and the taxane may be administered at a daily dose of about 0.1 mg to about 100 mg, such as about 40 mg to about 50 mg, wherein the daily doses of ritonavir and the taxane may be taken at the same or different time of day.

In another example, ritonavir may be administered at a weekly dose of about 50 mg to about 1200 mg, such as about 200 mg to about 400 mg, and the taxane may be administered at a weekly dose of about 30 mg to about 500 mg, such as about 40 mg to about 100 mg or about 40 mg to about 50 mg, wherein the weekly doses of ritonavir and the taxane may be taken on the same or different days.

In a fifth embodiment, the present invention provides a method for sensitizing or re-sensitizing cells to a taxane in the treatment of cancer, comprising administering an effective dose of ritonavir, or a pharmaceutically acceptable salt thereof.

The taxane may be selected from the group consisting of docetaxel, cabazitaxel, pharmaceutically acceptable salts or esters thereof, and combinations thereof. Preferably, the taxane is docetaxel. Other taxanes that are envisaged include paclitaxel.

In a sixth embodiment, the present invention provides ritonavir, or a pharmaceutically acceptable salt thereof, for use in sensitizing or re-sensitizing cells to a taxane in the treatment of cancer.

The taxane may be selected from the group consisting of docetaxel, cabazitaxel, pharmaceutically acceptable salts or esters thereof, and combinations thereof. Preferably, the taxane is docetaxel. Other taxanes that are envisaged include paclitaxel.

In a seventh embodiment, the present invention provides a kit comprising at least one pharmaceutical composition comprising a taxane and at least one pharmaceutical composition comprising a P-gp inhibitor, wherein said kit is for use in treating cancer in a taxane-resistant patient.

The taxane may be selected from the group consisting of docetaxel, cabazitaxel, pharmaceutically acceptable salts or esters thereof, and combinations thereof. Preferably, the taxane is docetaxel. Other taxanes that are envisaged include paclitaxel.

The P-gp inhibitor may be selected from the group consisting of ritonavir, elacridar and combinations thereof, or a pharmaceutically acceptable salt thereof. Preferably, the P-gp inhibitor is ritonavir.

The kit may comprise instructions for the simultaneous or sequential use of the pharmaceutical compositions for treating cancer in a taxane-resistant patient.

In an eighth embodiment, the present invention provides a method for the treatment of cancer, comprising a combination of a P-gp inhibitor and a taxane, comprising the steps of:
determining the expression of ABCB1 (P-gp) in a patient;
optionally, comparing the expression of ABCB1 (P-gp) to a reference level;
optionally, determining the P-gp inhibitor dosage based on the expression level of ABCB1 (P-gp) determined in the subject;
administering a combination of the P-gp inhibitor and the taxane, preferably at dosages based on the expression level of ABCB1 (P-gp) determined in the subject.

The taxane may be selected from the group consisting of docetaxel, cabazitaxel, pharmaceutically acceptable salts or esters thereof, and combinations thereof. Preferably, the taxane is docetaxel. Other taxanes that are envisaged include paclitaxel.

The P-gp inhibitor may be selected from the group consisting of ritonavir, elacridar and combinations thereof, or a pharmaceutically acceptable salt thereof. Preferably, the P-gp inhibitor is ritonavir.

Moreover, in any of the above embodiments of the invention, the following features may be provided.

Oral administration of the taxane and/or ritonavir (or other P-gp inhibitor) to a subject may include any route through the mouth that introduces or delivers to a subject the agent to perform its intended function. Suitable pharmaceutical compositions for oral administration includes liquids, tablets or capsules. Capsules and tablets may have an enteric coating, such that the taxane and/or ritonavir is released from the capsules or tablets in the intestine. Capsules and tablets may be formulated in an extended release formulation such that the taxane and/or ritonavir (or other P-gp inhibitor) is released over an extended period, e.g. several hours or more, e.g. during the time spend in the intestinal tract. Tablets and capsules may thus be formulated such that the agent is released therefrom gradually. Tablets and capsules may be formulated such that the agent is released in the stomach or intestine. Tablets and capsules may be formulated such that the agent is released in the stomach and intestine. Administration includes self-administration and the administration by another. Pharmaceutical compositions of this invention may comprise the taxane and/or ritonavir (or other P-gp inhibitor), or pharmaceutically acceptable salts and esters thereof, together with any pharmaceutically acceptable carrier, adjuvant or vehicle. Suitable preparations and/or pharmaceutical compositions for oral administrations include formulations as described in WO2009027644, WO2010020799 and Moes et al. Drug Deliv. Transl. Res. 2013) which are incorporated herein in their entirety by reference. Any suitable preparation for oral administration can be contemplated.

The current invention may not be restricted to oral administration of the taxane and/or ritonavir (or other P-gp inhibitor). Any administration of the taxane and/or ritonavir (or other P-gp inhibitor) via the gastrointestinal tract may be contemplated. Hence, enteral administration can be contemplated herein instead of oral administration. Preferably, enteral administration is in the form of capsules, tablets, and suppositories. Taxane and/or ritonavir (or other P-gp inhibitor) administration via a suppository may be advantageous, as bioavailability may be improved as compared with oral administration. This is because with oral administration, after passing the stomach and intestine, the taxane and/or ritonavir (or other P-gp inhibitor) is delivered to the liver via the portal vein. By enteral administration, the barriers that metabolize the taxane and/or ritonavir (or other P-gp inhibitor) in the first-pass may be avoided. Any enteral administration may suffice, as long as peak levels are avoided and effective plasma levels are obtained.

The taxane and ritonavir (or other P-gp inhibitor) can be administered simultaneously. It is understood that simultaneous administration can comprise administration of separate pharmaceutical preparations. For example, one pharmaceutical preparation suitable for oral administration comprising the taxane and another pharmaceutical preparation comprising ritonavir (or other P-gp inhibitor). The pharmaceutical preparation comprising ritonavir (or other P-gp inhibitor) preferably also being orally administered. It is understood that simultaneous administration can comprise one pharmaceutical preparation comprising both the taxane and ritonavir (or other P-gp inhibitor). Simultaneously, as used herein, means administration of the taxane or ritonavir (or other P-gp inhibitor) within e.g. approximately 20 minutes, more preferably within 15 minutes, more preferably within 10 minutes, even more preferably within 5 minutes, most preferably within 2 minutes of ritonavir (or other P-gp inhibitor) or the taxane. Generally, ritonavir (or other P-gp inhibitor) is preferably orally administered simultaneously with administering oral taxane as this provides for optimal compliance in self-administration by subjects receiving treatment.

Alternatively, the taxane and ritonavir (or other P-gp inhibitor) can be administered separately and/or sequentially from each other, preferably within approximately 60 minutes of each other. When they are administered separately, ritonavir (or other P-gp inhibitor) is preferably administered before the taxane, and, more preferably, within approximately 60 minutes before the taxane is administered.

A recommended dose of the taxane, such as docetaxel, may be between 75 mg/m² and 100 mg/m² every 3 weeks (milligrams of docetaxel per square meter of body surface area of a subject). A recommended dose for a non-small-cell lung cancer, a breast cancer, a gastric cancer, a head and neck cancer or a prostate cancer may be 75 mg/m² every 3 weeks. A recommended dose may also be 35 mg/m² per week. Taxane exposure levels of the tumour tissue can be defined herein as the area under the curve as obtained when administering the taxane intravenously and may correspond with an effective standard of care treatment for the taxane. It is understood that this may not define the actual taxane level of the tissue, as taxane is measured in plasma. The standard of care treatment for the taxane as used herein may be defined as an intravenous administration of a recommended dose of the taxane.

The area under the curve (AUC; ng*h/mL) is determined in the first 48 hours after the administration of the taxane, during which the taxane concentration in blood plasma can be measured at several timepoints, and the surface of the area under the curve can be calculated from the plotted values. Plasma levels of taxane can be measured by methods known in the art (Hendrikx et al. J. Chrom. B, 2011), which may include liquid chromatography and mass spectrometry methods. Plasma is a blood component, it is understood that instead of measuring the taxane in blood plasma, one can also determine levels of taxane in whole blood or in serum. Measurements of taxane, e.g. peak levels and area under the plasma concentration-time curve, in short area under the curve (AUC) herein are defined relative to (blood) plasma but can easily be recalculated to corresponding peak levels in whole blood or serum. For example, the AUC may be within the range of 500-2500 ng-h/mL, such as at least 500 ng-h/mL, at least 600 ng-h/mL, at least 800 ng-h/mL, at least 1000 ng-h/mL, or at least or 1200 ng-h/mL. For example, the AUC may be at most 2500 ng-h/mL, at most 2250 ng-h/mL, at most 2000 ng-h/mL, at most 1800 ng-h/mL, at most 1700 ng-h/mL, or at most 1500 ng-h/mL. For example, the AUC may be within the range of 800-1400 ng-h/mL. Herein, plasma concentration-time curve, area under the curve, or AUC, with reference to the taxane are used interchangeably and refer to the area under the curve in the first 48 hours (ng-h/mL) after the administration of the taxane. For example, these dosages may be reached by orally administering 50 mg of taxane, such as docetaxel, in two doses in one day (e.g. 30 mg morning and 20 mg evening) once a week.

Preferably, provided is the taxane or ritonavir (or other P-gp inhibitor) for use in accordance with the invention as described herein, wherein the cancer is a solid tumour. Preferably, said use of the taxane or ritonavir is a use wherein the solid tumour is a non-small-cell lung cancer, a gastric cancer, a breast cancer, a head and neck cancer or a prostate cancer. Said solid tumours being preferred because taxanes, such as docetaxel, have been shown to be highly efficacious in these cancers.

Most preferably, said cancer is a prostate cancer. The treatment of prostate cancer can involve the use of hormonal therapy, e.g. using androgen deprivation therapy. The prostate cancer may not respond to hormonal therapy, such prostate cancer termed hormonal refractory prostate cancer (HRPC). The prostate cancer may respond to hormonal therapy, such a prostate cancer termed hormonal sensitive prostate cancer (HSPC). Such patients may also have metastases, or may develop metastases during treatment. Such cancers referred to as mHRPC or mHSPC (m indicating metastatic). A prostate cancer treatment may include castration. In any case, prostate cancer generally involves the reduction of testosterone in the body to very low levels. As shown in the examples, in patients that have mCRPC, clearance of taxane as determined in plasma is apparently increased relative to patients having solid tumors that are not mCRPC. Such patients have very low levels of testosterone. Patients that receive intravenously administered taxane concurrent with androgen deprivation therapy in an early phase of the disease experience more toxicity relative to prostate cancer patients that receive intravenously administered taxane in a later phase of the disease. Hence, in prostate cancer treatments that involves the use of hormonal therapy, which involves androgen inhibitors, reduced taxane plasma levels by increased clearance of taxane can be anticipated as well, but such an increased clearance may need first to be established after hormonal therapy has commenced. The dosage of taxane and/or ritonavir (or other P-gp inhibitor) may therefore be adjusted in the early phase of the treatment of prostate cancer to compensate for relatively lower plasma levels in such patients as compared with patients with mCRPC. Conversely, plasma levels of taxane may need first to be established, e.g. by administering a first oral dosage of taxane combined with ritonavir (or other P-gp inhibitor) in accordance with the invention and determining plasma concentrations (such as AUC) of the taxane, to confirm that the same dosages as suitable for mCRPC can be administered. In any case, the dosages suitable for mCRPC may also be suitable for HSPC, HRPC, mHRPC, mHSPC or CRPC (i.e. non-metastatic). In a preferred embodiment, dosages suitable for mCRPC are also selected for the treatment of mHSPC.

In a preferred embodiment, the prostate cancer is a metastatic castration-resistant prostate cancer (mCRPC).

The dosing interval and/or dosage of the taxane and/or ritonavir (or other P-gp inhibitor) may be selected that allows to provide for desired taxane exposure levels in the tumour tissue which may be comparable or at least comparable to a standard of care treatment for docetaxel. For example, docetaxel or ritonavir (or other P-gp inhibitor) may be used in a combination therapy, where, docetaxel is administered orally in a weekly dosage, wherein the docetaxel exposure levels may be comparable to a standard of care treatment resulting in area under the curve of 600-1800 ng-h/mL, more preferably of 1000-1500 ng-h/mL.

In some embodiments, the weekly dose of ritonavir and/or the taxane may be split so that a subject takes, for example, a first dose in the morning and a second dose in the evening once a week. This may have the effect of decreasing the peak levels of the drug(s) in plasma which may aid in reducing side effects. It may also increase the time of systemic exposure of the drug. For example, ritonavir and/or the taxane can be administered on a bi-daily basis once a week, meaning that on one day every week, ritonavir and/or the taxane are administered twice, e.g. within an 8-16 hour interval. Accordingly, administrations of ritonavir and/or the taxane can be on a tri-daily, bi-daily or daily basis, every two days, weekly, every two weeks, every three weeks or any other suitable dosing interval. Combinations of these dosage regimens can also be used, for example, ritonavir and/or the taxane can be for bi-daily administration once every week or every two or three weeks.

For example, in an embodiment, for the treatment of solid tumors, docetaxel is administered orally at a weekly dosage of 50 mg. Such an administration is preferably at a bi-daily weekly schedule. Hence, a bi-daily weekly schedule for the treatment of solid tumors is provided herein for the treatment of solid tumors, wherein docetaxel is administered on one day a week a first administration at a dosage of 30 mg docetaxel with 100 mg ritonavir and a second administration at a dosage of 20 mg docetaxel with 100 mg ritonavir.

In another embodiment, for the treatment of solid tumors, docetaxel is administered orally at a weekly dosage of 40 mg. Such an administration is preferably at a bi-daily weekly schedule. Hence, a bi-daily weekly schedule for the treatment of solid tumors is provided herein for the treatment of solid tumors, wherein docetaxel is administered on one day a week a first administration at a dosage of 20 mg docetaxel with 200 mg ritonavir and a second administration at a dosage of 20 mg docetaxel with 100 mg ritonavir.

In one embodiment, a bidaily weekly schedule is provided for the treatment of cancer, wherein docetaxel is administered on one day a week a first administration at a dosage of 30 mg docetaxel with 200 mg ritonavir and a second administration at a dosage of 20 mg docetaxel with 200 mg ritonavir. In another embodiment, a bidaily weekly schedule is provided for the treatment of cancer, wherein docetaxel is administered on one day a week a first administration at a dosage of 20 mg docetaxel with 200 mg ritonavir and a second administration at a dosage of 20 mg docetaxel with 200 mg ritonavir. In still another embodiment, a bidaily weekly schedule is provided for the treatment of cancer, wherein docetaxel is administered on one day a week a first administration at a dosage of 20 mg docetaxel with 100 mg ritonavir and a second administration at a dosage of 20 mg docetaxel with 100 mg ritonavir. In another embodiment, a bidaily weekly schedule is provided for the treatment of cancer, wherein docetaxel is administered on one day a week a first administration at a dosage of 20 mg docetaxel with 200 mg ritonavir and a second administration at a dosage of 20 mg docetaxel with 100 mg ritonavir.

In a further embodiment, docetaxel is administered orally in the treatment of mCRPC at weekly dosage of 50 mg. Such an administration is preferably at a bidaily weekly schedule. The ritonavir dosage may need to be adapted for mCRPC patients such that the defined AUC could be obtained. Hence, a bidaily weekly schedule for the treatment of mCRPC is provided herein for the treatment of cancer, wherein docetaxel is orally administered on the same day a first administration at a dosage of 30 mg docetaxel with 200 mg ritonavir and a second administration at a dosage of 20 mg docetaxel with 100 mg ritonavir.

Taxane levels in subjects may be controlled before treatment and/or monitored and controlled in treatment following the use of oral administration of the taxane and ritonavir (or other P-gp inhibitor). Such monitoring and control may alternatively (or additionally) also be exerted by measuring the taxane in blood plasma. Such monitoring and control may also be exerted by monitoring side effects. Taxane clearance in a subject may vary due to unknown causes. Hence, monitoring taxane levels in subjects during treatment allows one to adapt the taxane dosages to maintain appropriate levels of taxane in the subjects. Monitoring side effects assists therein as well. When an area under the curve is determined, one can determine the suitable dosages of ritonavir (or other P-gp inhibitor) and the taxane combination that may achieve the desired exposure levels of tumour tissue. For example, in a first adaptation, the area under the curve increase may be too high, resulting in undesired side effects. Upon a second adaptation, the defined area under the curve may be achieved, providing for the defined exposure levels of tumour tissue while having significantly reduced side effects.

Accordingly, the invention may comprise the steps of:
administering a combination of the taxane and ritonavir (or other P-gp inhibitor);
determining plasma levels of the taxane in the subject;
optionally, comparing the levels of the taxane to a reference level;
determining the taxane dosage for administration of a subsequent combination of ritonavir (or other P-gp inhibitor) and the taxane; and
administering the subsequent combination of the ritonavir (or other P-gp inhibitor) and the taxane.

By determining the plasma levels of taxane after a first administration, it can be confirmed that the selected dosages of the first administration (of both the ritonavir (or other P-gp inhibitor) and the taxane) are suitable dosages. Conversely, when plasma levels are too high or low, the dosages of the subsequent combination of ritonavir (or other P-gp inhibitor) and the taxane may be adjusted. Either ritonavir (or other P-gp inhibitor) or the taxane dosage or both ritonavir (or other P-gp inhibitor) and the taxane dosage may be adjusted. Hence, the ritonavir (or other P-gp inhibitor) dosage may remain the same at the first administration and subsequent dosages, and the taxane dosage adjusted to compensate for any increase or decrease in taxane plasma level. Accordingly, in such a method of treatment involving determining taxane plasma levels, ritonavir (or other P-gp inhibitor) is administered at a pre-determined dosage. Also, the taxane dosage may remain the same at the first administration and subsequent dosages and the ritonavir (or other P-gp inhibitor) dosage adjusted to compensate for any increase or decrease in taxane plasma level. Preferably, the dosage of the taxane is to be adjusted in order to eradicate the tumour cells.

This way, throughout the treatment, the dosage of the taxane of at least the subsequent combination of ritonavir (or other P-gp inhibitor) and the taxane may be sufficient to obtain desired taxane exposure levels of the tumour tissue, which may be comparable or at least comparable to a standard of care treatment for the taxane. Preferably, the method for the treatment of a cancer comprises multiple administrations of a combination of ritonavir (or other P-gp inhibitor) and the taxane, wherein after each administration the levels of the taxane are determined, for determining the taxane dosage for administration of a subsequent combination of ritonavir (or other P-gp inhibitor) and the taxane. Hence, when the level of taxane is increased in the subject as compared with the reference level, the dosage of taxane is reduced, and wherein when the level of taxane is decreased during treatment as compared with a reference level, the dosage of taxane is increased, as compared with the previous dosage administered.

In another embodiment, the invention may comprise the steps of:
determining the expression of ritonavir (or other P-gp inhibitor) in a subject;
optionally, comparing the expression of ritonavir (or other P-gp inhibitor) to a reference level;
determining the ritonavir (or other P-gp inhibitor) dosage based on the expression level of ritonavir determined in the subject;
administering a combination of ritonavir (or other P-gp inhibitor) at the determined dosage and the taxane,
wherein the dosage of taxane is sufficient to obtain taxane exposure levels of the tumour tissue comparable to a standard of care treatment for the taxane.

As the expression of ritonavir (or other P-gp inhibitor) in a subject may have an effect on the dosage of taxane that is to be administered to obtain sufficient exposure levels, determining the expression of ritonavir (or other P-gp inhibitor) may have the advantage that possible variation between subjects can be taken into account. This can be done before the treatment commences. The ritonavir (or other P-gp inhibitor) expression in a subject can be determined by any known means, but also by measuring plasma levels of ritonavir (or other P-gp inhibitor) or other indirect methods. By knowing before treatment commences what an appropriate dose of ritonavir (or other P-gp inhibitor) is, from the initiation of treatment, immediately the desired exposure levels of docetaxel in the subject can be obtained. Hence, in a preferred embodiment, in a method in accordance with the invention the steps of determining the expression, subsequent optional comparison step, and determining dosage step for ritonavir (or other P-gp inhibitor), are carried out prior to the first administration of the combination of ritonavir (or other P-gp inhibitor) and the taxane.

In further embodiments, kits are provided that are for use in the methods and uses as described herein for the combinations of the taxane and a P-gp inhibitor, such as ritonavir or elacridar. In one embodiment, a kit is provided comprising a pharmaceutical composition comprising the taxane for oral administration and a pharmaceutical composition comprising a P-gp inhibitor, such as ritonavir or elacridar. In another embodiment, a kit is provided comprising a pharmaceutical composition comprising the taxane for oral administration and a pharmaceutical composition comprising a P-gp inhibitor, such as ritonavir or elacridar, wherein said kit is for the treatment of a solid tumour, in particular non-small cell-lung cancer, gastric cancer, a breast cancer, a head and neck cancer or a prostate cancer, more in particular mCRPC. In still another embodiment, a kit is provided comprising a pharmaceutical composition comprising the taxane and a pharmaceutical composition comprising a P-gp inhibitor, such as ritonavir or elacridar, wherein said kit is for use in a combination therapy as defined in any one of the methods and uses in accordance with the invention as described herein.

Pharmaceutical compositions of this invention may comprise the taxane, or pharmaceutically acceptable salts and esters thereof, and/or ritonavir (or other P-gp inhibitor) or pharmaceutically acceptable salts and esters thereof together with any pharmaceutically acceptable carrier, adjuvant or vehicle.

As used in the description of the invention, clauses and clauses appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”). As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

### EXAMPLES

### Summary

In a first study, the protein expression levels of P-gp in parental and docetaxel resistant prostate cancer cell lines (DU-145, DU-145 DOC10, 22Rv1 and 22Rv1 DOC8) was assessed by western blot analysis. The study consisted of 5 parts. In the first part the protein expression levels of P-gp in the parental and docetaxel resistant cell lines was assessed by western blot analysis. Next, the anti-cancer activity of cabazitaxel in monotherapy against the four cell lines was assessed using a 3D clonogenic assay to determine the concentrations for the subsequent combination studies. The anti-cancer activity of different two-drug combinations: docetaxel+elacridar; docetaxel+ritonavir; and cabazitaxel+ritonavir, was investigated using a 3D clonogenic assay in an 8*2 matrix layout combining 8 concentrations of the first compound with 2 concentrations of the second compound. Colony count based on image analysis was used as read-out. The Bliss independence methodology was used for data analysis, in order to identify additive/synergistic/antagonistic effects. A clear upregulation in P-gp (MDR1) protein expression in the resistant cell lines was observed, compared to the corresponding parental lines. This is in line with the results of a previous study, in which both resistant cell lines were shown to overexpress mdr-1 mRNA as determined by RNA-Seq.

Ritonavir and elacridar have been shown to inhibit P-gp and were tested in combination with docetaxel to examine their docetaxel resistance-reverting effect. At a concentration of 31.6 μM ritonavir decreased the relative IC₅₀ value of docetaxel from 0.219 μM to 0.0026 μM (84-fold) and strong synergistic effects were observed at mid concentrations of docetaxel. 100 μM ritonavir inhibited tumour colony formation completely and no additional effects could be seen in the combination with docetaxel.

For a combination of docetaxel+/−elacridar, synergistic effects were observed in the docetaxel resistant cell lines 22Rv1 DOC8 and DU-145 DOC10 but not in the parental cell line DU-145. Thus, the effect of ritonavir that was observed in previous studies could be reproduced by elacridar, strongly suggesting that inhibition of P-gp is responsible for the resistance-reverting effect.

Next, the anti-cancer activity of cabazitaxel in monotherapy against the four cell lines was investigated using a 3D clonogenic assay to determine the IC₅₀ concentrations. Cabazitaxel inhibited tumour cell growth in a concentration dependent manner in all four cell lines with higher IC₅₀ values in the docetaxel resistant compared to the parental cell lines. For 22Rv1 a 14.5-fold higher relative IC₅₀ value compared to 22Rv1 was observed. For DU-145 DOC8 vs. DU-145 the shift of the IC₅₀ value was 2.7-fold. Thus, resistance to docetaxel also resulted in lower sensitivity towards cabazitaxel.

For the combination of cabazitaxel+/−ritonavir, strong synergistic effects were observed in the docetaxel resistant cell line 22Rv1 DOC8, whereas in the parental cell lines and DU-145 DOC10 no or only low synergistic effects were observed. In 22Rv1 DOC8, the resistance to cabazitaxel was completely reverted by ritonavir.

### Experimental Methods

For the single agent efficacy experiment, a master stock solution of cabazitaxel was prepared by dissolving compound powder in DMSO at a concentration of 10 mM. From this a working stock solution of 0.1 mM (330-fold the highest test concentration) was prepared. Small aliquots of this solution were stored at −20° C. On each day of an experiment, a frozen aliquot of the working stock solution was thawed and stored at room temperature prior to and during treatment. First, serial half-log dilutions of the working stock solution were prepared in DMSO. These dilutions were then diluted 1:22 into cell culture medium in an intermediate dilution plate. Finally, 10 μl taken from the intermediate dilution plate were transferred to 140 μl/well of the final assay plate. Thus, the DMSO working stock was diluted 1:330, and the DMSO concentration in the assay was 0.3% v/v.

For combination experiments, working stock solutions of docetaxel, ritonavir, elacridar, and cabazitaxel were prepared in DMSO at concentrations of 660-fold the highest test concentration used. Small aliquots of this stock solution were stored at −20° C. On each day of an experiment, a frozen aliquot of the working stock solutions was thawed and stored at room temperature prior to and during treatment. First, 1:2 dilutions of 660-fold concentrated working stocks were done in DMSO (in case of monotherapy controls), or equal amounts of the compound working stocks were mixed (in case of drug combinations). Then, different dilutions of these 330-fold concentrated solutions were done in DMSO to give a 8*2 matrix combination layout. The DMSO dilutions were further diluted 1:22 into cell culture medium (IMDM, supplemented with 20% (v/v) fetal calf serum, 50 μg/ml gentamicin) in an intermediate dilution plate. Finally, 10 μl taken from the intermediate dilution plate were transferred to 140 μl of cells and medium per well of the final assay plate. Thus, the DMSO dilutions used for the mono- and the combination-treatment were diluted 1:330, and the DMSO concentration in the assay was 0.3% v/v.

DU-145 DOC10 was generated from DU-145 and 22Rv1 DOC8 was generated from 22Rv1. 22Rv1 was obtained from American Type Culture Collection (ATCC, Rockville, MD, USA), whereas DU-145 was obtained from National Cancer Institute (NCI, Bethesda, MD, USA).

Tumour cell lines were grown at 37° C. in a humidified atmosphere with 5% CO2 in RPMI 1640 medium (Biochrom) supplemented with 10% (v/v) fetal calf serum and 50 μg/ml gentamicin. Docetaxel resistant sublines were cultured in the presence of 8 nM (22Rv1) or 10 nM docetaxel (DU-145). The cells were routinely passaged once or twice weekly and maintained in culture for up to 20 passages. For the assay, cells were harvested using TrypLE and the percentage of viable cells was determined by the CASY TT cell counter (OMNI Life Science).

The clonogenic assay was carried out in a 96 well plate format using ultra low attachment plates. For each test, cells were prepared and assay plates were prepared as follows: each test well contained a layer of semi-solid medium with tumour cells (50 μl), and a second layer of medium supernatant with or without test compound (100 μl). The cell layer consisted of 2·10³ to 3·10³ tumour cells per well, which were seeded in 50 μl/well cell culture medium (IMDM, supplemented with 20% (v/v) fetal calf serum, 50 μg/ml gentamicin, and 0.4% (w/v) agar). After 24 h, the soft-agar layer was covered with 90 μl of the same culture medium without agar, and 10 μl of control medium or test compound after serial dilution in DMSO and transfer in cell culture medium were added, and left on the cells for the duration of the experiment (continuous exposure, 100 μl drug overlay). Every plate included six untreated control wells and drug-treated groups. Duplicate values were obtained by testing single values per condition on one plate. Cultures were incubated at 37° C. and 7.5% CO₂ in a humidified atmosphere for 13 days and monitored closely for colony growth using an inverted microscope. Within this period, tumour growth led to the formation of colonies with a diameter of >50 μm (area >2000 μm²). At the time of maximum colony formation, vital colonies were stained for 48 hours with a sterile aqueous solution of 2-(4-iodophenyl)-3-(4-nitrophenyl)-5-phenyltetrazolium chloride (INT, 1 mg/ml, 25 μl/well), and colony counts were performed with an automatic image analysis system (Celllnsight NXT, Thermo Scientific or Bioreader 5000 V-alpha, BIO-SYS GmbH).

### Results and Discussion

As shown in FIG. 1, acquired resistance was observed in DU-145 prostate cancer cells, that were exposed to docetaxel for 3 months, as demonstrated by an ˜20-fold increase in IC₅₀ value compared to the parental, untreated cells. The resistance of the cells was confirmed in a 3D culture (clonogenic assay) setup. Resistance to docetaxel is more pronounced in the 2D setup (21-fold) than in the 3D setup (3.3-fold).

As shown in FIG. 2, acquired resistance was observed in 22Rv1 prostate cancer cells, that were exposed to docetaxel for 3 months, as demonstrated by a >100-fold increase in IC₅₀ value compared to the parental, untreated cells. The resistance of the cells was confirmed in a 3D culture (clonogenic assay) setup. The resistance to docetaxel is more pronounced in the 2D setup (105-fold) than in the 3D setup (42-fold).

As shown in FIG. 3, high P-glycoprotein (P-gp protein) overexpression is observed in docetaxel resistant prostate cancer calls. Very high P-gp protein overexpression is observed in DU-145 DOC10 cells. P-pg protein overexpression is also observed in 22RV1 DOC8 cells. Also, high P-gp (MDR-1) mRNA overexpression is observed in DU-145 DOC10 cells (3287-fold compared to parent) and in 22Rv1 DOC8 cells (2772-fold overexpression). 22RV1 DOC8 cells express less P-gp protein than DU-145 DOC10, even though their resistance to docetaxel is much higher (134-fold vs 22-fold, respectively in 2D culture conditions). This also suggests that ab initio resistance to taxanes may be present in patients who have naturally higher expression or overexpression of P-pg protein.

As shown in FIG. 4, in both drug resistant cell lines DU-1245 DOC10 and 22Rv1 DOC8, the gene which was upregulated most was ABCB1 (P-glycoprotein) (3287-fold in DU145 DOC10 and 2773-fold in 22Rv1 DOC8). This also suggests that ab initio resistance to taxanes may be present in patients who have naturally higher expression or overexpression of P-pg protein.

It was therefore concluded that docetaxel resistance in the DU-145 and 22Rv1 prostate cancer cell lines appears to be driven largely by distinct gene expression and functional pathway responses. A degree of overlap was identified in the responses of the two cell lines which may indicate common resistance-associated mechanisms. In both cell lines, P-glycoprotein was the highest overexpressed gene.

Ritonavir is an inhibitor of P-glycoprotein. As shown in FIG. 5 and the table below, ritonavir reverses docetaxel resistance in castration-resistant DU-145 DOC10 prostate cancer cells (clonogenic assay). 10 μM ritonavir induced an increase in sensitivity to docetaxel (3.4-fold lower IC₅₀ value) in DU-145 DOC10 resistant cells. The addition of 10 μM ritonavir to the resistant cells resulted in a sensitivity (IC₅₀=2.8 nM) identical to that of the parental cells (IC₅₀=2.9 nM), demonstrating a reversal of resistance.

| | |
|---|---|
| DU-145 | DU-145 DOC10 |

| | | |
|---|---|---|
| Ritonavir | IC₅₀ value in nM | Relative Resistance* |

| | | | |
|---|---|---|---|
| — | 2.9 | 9.5 | 3.3 |

| | | | | |
|---|---|---|---|---|
| 1 | μM | 1.8 | 8.8 | 3.0 |
| 10 | μM | 1.7 | 2.8 | 1.0 |

*Compared to parental DU-145 cells without ritonavir

As shown in FIG. 6, ritonavir and docetaxel acted synergistically in docetaxel resistant DU-145 DOC10 prostate cancer cells (clonogenic assay). 10 μM ritonavir induced an increase in sensitivity to docetaxel (3.4-fold lower IC₅₀ value) in DU-145 DOC10 resistant cells. The combination of 10 μM ritonavir with 3 nM docetaxel resulted in a strong synergistic effect in DU-145 DOC10 resistant cells, in contrast to parental cells.

As shown in FIG. 7 and the table below, ritonavir also reverses docetaxel resistance in 22Rv1 DOC8 resistant prostate cancer cells. In the resistant cells, strong synergistic effects were observed in the combination of 32 μM ritonavir and mid concentrations of docetaxel (3.16 nM to 316 nM), demonstrating that resistance could be reverted by ritonavir.

| 22Rv1 DOC8 | Docetaxel |
|---|---|
| Ritonavir | IC₅₀ value in nM |
| — | 202 |
| 32 μM | 3 |

As shown in FIG. 8, ritonavir and cabazitaxel induce synergistic effects in parent DU-145 prostate cancer cells. Ritonavir inhibits the growth of DU145 at concentrations of 10 μM and higher. The combination of 10 μM ritonavir and 0.3 nM cabazitaxel has a synergistic effect in DU145 cells.

As shown in FIG. 9, ritonavir and cabazitaxel induce synergistic effects in docetaxel-resistant DU-145 DOC10 prostate cancer cells. Ritonavir inhibits the growth of DU145 DOC10 at concentrations of 10 μM and higher. The combination of 10 μM ritonavir and 1 nM cabazitaxel has a synergistic effect in DU-145 DOC10 resistant cells.

As shown in FIG. 10 and the table below, ritonavir reverses cabazitaxel resistance in docetaxel-resistant DU145 DOC10 prostate cancer cells. 10 μM ritonavir induced an increase in sensitivity to docetaxel (2.5-fold lower IC₅₀ value) in DU-145 DOC10 resistant cells. The addition of 10 μM ritonavir to the resistant cells resulted in a sensitivity (IC₅₀=0.6 nM) identical to that of the parental cells (IC₅₀=0.6 nM), demonstrating a reversal of resistance.

| | |
|---|---|
| DU145 | DU145 DOC10 |

| | | |
|---|---|---|
| Ritonavir | IC₅₀ value in nM | Relative Resistance* |

| | | | |
|---|---|---|---|
| — | 0.6 | 1.5 | 2.5 |
| 10 μM | 0.3 | 0.6 | 1 |

*Compared to parental DU-145 cells without ritonavir

As shown in FIG. 11, ritonavir and cabazitaxel induced synergistic effects in docetaxel-resistant 22Rv1 DOC8 prostate cancer cells. The combination of 32 μM ritonavir and 1, 3 and 10 nM cabazitaxel results in a synergistic effect in 22Rv1 DOC8 resistant cells.

As shown in FIG. 12 and the table below, ritonavir reverses cabazitaxel resistance in docetaxel-resistant 22Rv1 DOC8 prostate cancer cells. 32 μM ritonavir induced an increase in sensitivity to docetaxel (14-fold lower IC₅₀ value) in 22Rv1 DOC8 resistant cells. The addition of 32 μM ritonavir to the resistant cells resulted in a sensitivity (IC₅₀=0.7 nM) identical to that of the parental cells (IC₅₀=0.7 nM), demonstrating a reversal of resistance.

| | |
|---|---|
| 22Rv1 | 22Rv1 DOC8 |

| | | |
|---|---|---|
| Ritonavir | IC₅₀ value in nM | Relative Resistance* |

| | | | |
|---|---|---|---|
| — | 0.7 | 9.5 | 14 |
| 10 μM | 0.7 | 5.2 | |
| 32 μM | 0.7 | 0.7 | |

*Compared to parental 22Rv1 cells without ritonavir

As shown in FIG. 13 and the table below, elacridar reverses docetaxel resistance in castration-resistant DU-145 DOC10 prostate cancer cells. 0.3 and 1 μM elacridar induced an increase in sensitivity to docetaxel (6- to 7-fold lower IC₅₀ value) in DU-145 DOC10 resistant cells. The addition of 0.3 and 1 μM elacridar to the resistant cells resulted in a sensitivity (IC₅₀=1.9 and 1.6 nM) almost identical to that of the parental cells (IC₅₀=1.1 nM), demonstrating a reversal of resistance.

| | |
|---|---|
| DU-145 | DU-145 DOC10 |

| | | |
|---|---|---|
| Elacridar | IC₅₀ value in nM | Relative Resist* |

| | | | |
|---|---|---|---|
| — | 1.1 | 11.7 | 11 |

| | | | | |
|---|---|---|---|---|
| 0.3 | μM | 1.1 | 1.9 | 1.7 |
| 1 | μM | 1.1 | 1.6 | 1.5 |

*Compared to parental DU-145 cells without elacridar

As shown in FIG. 14, elacridar and docetaxel were synergistic in docetaxel resistant DU-145 DOC10 prostate cancer cells (clonogenic assay). The combination of 0.3 or 10 μM elacridar with 3 or 10 nM docetaxel resulted in a strong synergistic effect in DU-145 DOC10 resistant cells. No synergistic effect was observed in the parental cells DU-145 cells.

As shown in FIG. 15 and the table below, elacridar reverses docetaxel resistance in castration-resistant 22Rv1 DOC8 prostate cancer cells. 0.3 and 1 μM elacridar induced an increase in sensitivity to docetaxel (100-fold lower IC₅₀ value) in 22Rv1 DOC8 resistant cells. The addition of 0.3 and 1 μM elacridar to the resistant cells resulted in a sensitivity (IC₅₀=1.4 and 1.3 nM) identical to that of the parental cells (IC₅₀=1.3 nM), demonstrating a reversal of resistance.

| | |
|---|---|
| 22Rv1 | 22Rv1 DOC8 |

| | | |
|---|---|---|
| Elacridar | IC₅₀ value in nM | Relative Resist** |

| | | | |
|---|---|---|---|
| — | 1.3* | 134 | 103 |

| | | | | |
|---|---|---|---|---|
| 0.3 | μM | Not done | 1.4 | 1.1 |
| 1 | μM | Not done | 1.3 | 1 |

*IC50 values from previously performed experiment
**Compared to parental 22Rv1 cells without elacridar

The above embodiments have been described by way of example only. Many other embodiments falling within the scope of the accompanying claims will be apparent to the skilled reader. Therefore, although the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

Any feature that has been described above in relation to any one aspect or embodiment of the invention is also disclosed hereby in relation to all other aspects and embodiments. Likewise, all combinations of two or more of the individual features or elements described above may be present in any aspect or embodiment. For brevity, all possible features and combinations have not been recited in relation to all aspects and embodiments, but they are expressly contemplated and hereby disclosed.

1. A method for treating cancer in a taxane-resistant patient, the method comprising administering an effective dose of a taxane simultaneously or sequentially with ritonavir or a pharmaceutically acceptable salt thereof.

2. A method for reversing taxane resistance in a patient, comprising administering an effective dose of ritonavir or a pharmaceutically acceptable salt thereof.

3. The method according to claim 2, wherein ritonavir, or a pharmaceutically acceptable salt thereof, is administered simultaneously or sequentially with an effective dose of a taxane.

4. The method according to claim 1, wherein the cancer comprises a solid tumour.

5. The method according to claim 1, wherein the cancer is selected from the group consisting of gastric cancer, breast cancer, head and neck cancer, lung cancer, and prostate cancer.

6. (canceled)

7. The method according to claim 5, further comprising determining if the cancer is responsive to treatment by determining the responsiveness of a DU-145 or 22Rv1 cell line to an effective dose of a taxane simultaneously or sequentially with ritonavir or a pharmaceutically acceptable salt thereof.

8. The method according to claim 1, wherein ritonavir and/or a taxane is administered orally.

9. The method according to claim 1, wherein ritonavir and/or a taxane is administered intravenously.

10. The method according to claim 1, wherein the taxane is selected from the group consisting of docetaxel, cabazitaxel, pharmaceutically acceptable salts or esters thereof, and combinations thereof.

11. The method according to claim 1, wherein the taxane is docetaxel, or a pharmaceutically acceptable salt or ester thereof.

12. The method according to claim 1, wherein ritonavir is administered at a weekly dose of about 50 mg to about 1200 mg.

13. The method according to claim 1, wherein ritonavir is administered at a daily dose of about 50 mg to about 1200 mg.

14. The method according to claim 1, wherein the taxane is administered at a weekly dose of about 30 mg to about 500 mg.

15. The method according to claim 1, wherein the taxane is administered at a daily dose of about 0.1 mg to about 100 mg.

16. The method according to claim 1, wherein ritonavir is administered at a daily dose of about 50 mg to about 1200 mg and the taxane is administered at a daily dose of about 0.1 mg to about 100 mg, wherein the daily doses of ritonavir and the taxane may be taken at the same or different time of day.

17. The method according to claim 1, wherein ritonavir is administered at a weekly dose of about 50 mg to about 1200 mg and the taxane is administered at a weekly dose of about 30 mg to about 500 mg, wherein the weekly doses of ritonavir and the taxane may be taken on the same or different days.

18.-53. (canceled)

54. A kit comprising:
at least one pharmaceutical composition comprising a taxane; and
at least one pharmaceutical composition comprising a P-gp inhibitor.

55. The kit of claim 54, wherein the taxane is selected from the group consisting of docetaxel, cabazitaxel, pharmaceutically acceptable salts or esters thereof, and combinations thereof.

56. The kit of claim 54, where the P-gp inhibitor is selected from the group consisting of ritonavir, elacridar and combinations thereof, or a pharmaceutically acceptable salt thereof.

57. (canceled)

58. The method according to claim 1, further comprising:
determining the expression of ABCB1 (P-gp) in the patient;
optionally, comparing the expression of ABCB1 (P-gp) to a reference level; and
optionally, determining a dosage of the ritonavir based on the expression level of ABCB1 (P-gp) determined in the subject.

59.-60. (canceled)
